(19) United States
(12) Patent Application Publication
(10) Pub. No.: US 2020/0110620 A1
(43) Pub. Date: Apr. 9, 2020

(54) SYSTEM AND METHOD OF MONITORING AND REPORTING EQUIPMENT STATUS

(71) Applicant: Summit Imaging, Inc., Woodinville, WA (US)

(72) Inventor: Lawrence R. Nguyen, Woodinville, WA (US)

(21) Appl. No.: 16/594,859

(22) Filed: Oct. 7, 2019

Related U.S. Application Data
(60) Provisional application No. 62/742,028, filed on Oct. 5, 2018.

(51) Int. Cl. G06F 9/445; G16H 40/40; G06F 21/57; G16H 10/60; G06F 8/61; G06F 21/33

(52) U.S. Cl. CPC G06F 9/44505; G16H 40/40; G06F 21/33; G16H 10/60; G06F 8/61; G06F 21/575

(57) ABSTRACT

A computer-implemented tool for use with an medical device having a computer system, the tool including an unsecured software-implemented suite of diagnostic tools and utilities configured to read logs, read system configuration, provide a Telnet function, eject a USB, and deactivate itself, and a secured software-implemented suite of diagnostic tools and utilities configured to interpret a key file received from an external source, upload the tool into the computer system of the medical device, run the tool upon booting up of the computer system to enable a replacement component in the medical device to be activated after installation in the medical device.

### BACKGROUND

### Technical Field

The present disclosure pertains to the maintenance of electronic equipment and, more particularly, to enabling remote reporting of the functional status of hardware and remote access to a compromised component for resolving errors and allowing activation following reinstallation of the repaired component in the electronic equipment.

### Description of the Related Art

Modern electronic equipment employs numerous parts and components, such as circuit boards, that are interconnected and configured to carry out desired functions. Over time, components will require service and replacement.

Determining the status of components in electronic equipment, particularly medical equipment, and identifying risks of failures as well as actual failures and identifying components that have failed or are in the process of failing enables owners and technicians to efficiently maintain and repair the equipment.

Moreover, to ensure the electronic equipment functions properly, it is necessary to ensure that replacement components are the correct components and are authorized for installation and use on the equipment. Authorization can take several forms, including authorization through OEM personnel as well as automated authorization that takes place on the system itself, and a combination of authorization obtained from a technician or from the organization that owns the equipment of an independent service organization that is contracted by the organization that owns the equipment and via the automated system.

To this end, the electronic equipment is typically configured to recognize and permit operation of only authorized components. When a refurbished or replacement component is not recognized by the equipment's controller upon start-up, the controller will block the operation of the unrecognized component and, in most cases, generate an error code that is sent to an operator of the equipment.

In some situations a genuine replacement or refurbished component will not be automatically activated by the equipment. This can cause serious problems for the owner or user of the equipment, especially in the medical field. Equipment downtime can be costly to businesses and severely impair the ability of service entities to render services to those in need. Moreover, many customers have employees and technicians on staff to service the various devices used in diagnostics and evaluation of patients. For example, medical facilities such as hospitals service their own equipment and need the capability to quickly bring a machine back online. It is an FDA regulation that purchasers of such equipment be enabled to service their own equipment, but there are many road blocks through the OEMs to doing this. Hence, there is a need for a process that allows the operator to ensure the replacement component is activated by the equipment to resume normal operation.

### BRIEF SUMMARY

The present disclosure is directed to the monitoring, maintenance, and repair of electronic equipment, including medical devices, such as medical imaging equipment. A system and method are provided to facilitate remote monitoring and reporting of equipment and component operating conditions, risk of failure, and actual failure as well as complete repair, such as component replacement and reinstatement of the equipment to operational status.

The system and method include a process configured to remotely identify and save error codes and equipment data and logs in the medical device. Ideally critical equipment logs are accessed, the data copied without altering the data and the equipment, and the data is then saved to an onsite or offsite location.

In accordance with another aspect of the present disclosure, the system and method includes a software module to access equipment logs on equipment, to obtain data regarding equipment operation and condition, and distribute the data to a user interface.

In accordance with another aspect of the present disclosure, the system and method utilizes a file transfer protocol to distribute the data to the user interface.

In accordance with another aspect of the present disclosure, the system and method include a software tool saved on a portable memory device to be executed when connected to the equipment to provide access to equipment logs, to obtain data regarding equipment operation and equipment condition, and transfer the data to a user interface.

In accordance with another aspect of the present disclosure, the system and method can include clearing the identified error codes, and activating a replacement component associated with the identified error codes in the medical device using external service tools. This includes reading errors and logs in the system to ensure it is returned to OEM specifications as required by law.

In accordance with one aspect of the present disclosure, a computer-implemented tool for use with a medical device having a computer system is provided. The tool includes executable code stored on a portable database configured to access equipment logs; an unsecured software-implemented suite of diagnostic tools and utilities configured to read error logs, read a build of the data and information, provide a Telnet or FTP function, eject a USB, and to deactivate itself; a secured software-implemented suite of diagnostic tools and utilities configured to interpret a key file received from an external source; and upload and run upon booting up of the computer system of the medical device based on the key file, enable a replacement component in the medical device to be activated after installation in the medical device, and then delete or uninstall itself to restore a file structure back to OEM standards.

In accordance with another aspect of the present disclosure, the tool includes a communication circuit configured to communicate with the external source to receive the key file.

In accordance with yet another aspect of the present disclosure, a system is provided that includes a medical device having a software tool saved on a portable memory device coupled to the medical device, the software tool to be executed when connected to the equipment to provide access to equipment logs, to obtain data regarding equipment operation and equipment condition, and transfer the data to a user interface.

In accordance with another aspect of the present disclosure, a controller is provided with a control circuit and a memory in communication with the control circuit; a plurality of components in communication with the controller, the plurality of components including at least one replacement component; and a remote device capable of being in communication with the memory of the medical device and configured to communicate with the memory to enable the medical device to activate the at least one replacement component. The remote device includes a memory storage device having stored thereon a plurality of unsecured diagnostic and utility programs and a plurality of secured diagnostic and utility programs that are configured to be activated upon receipt of one or more key files; and software configured to receive the one or more key files to enable the use of secured functions of the secured diagnostic and utility programs to provide an enabling code to the medical device to be used to activate the at least one replacement component.

In accordance with still yet another aspect of the present disclosure, the system includes a remote provider configured to communicate with the remote device and provide the one or more key files to the remote device.

In accordance with a further aspect of the present disclosure, a method is provided for activating a component in a medical device, the method including the steps of: receiving a key file from an external source; uploading a protocol to a memory of the medical device; running the protocol as enabled by the key file, the protocol configured to enable the activation of one or more replacement components; and removing the protocol from the memory of the medical device after the one or more replacement components are activated.

In accordance with a further aspect of the present disclosure, the method further includes: reading an error log to identify and interpret error codes in the error log, identifying one or more replacement components in the medical device that are not activated, and clearing the error log based on the key file.

In accordance with yet another aspect of the present disclosure, a method is provided that includes accessing an operating system on an electronic equipment, configuring the operating system to run an executable program when triggered by one or more events, executing the executable program to access equipment logs, and copying equipment log information to a second location for display on a user interface.

The advantages of the system and method of the present disclosure include very rapid response to malfunctions and error codes, lower downtime to mission critical equipment, and much lower cost to the user or owner of the equipment. Replacement components can be obtained for a fraction of the cost of OEM components, and this eliminates the need to outsource service calls. It is configurable within existing IT infrastructure, uses current access controls, and in the medical field it is HIPAA compliant as well as GDRP ready. The system and method provide single point access for data to be distributed to the cloud, and is fully configurable and customizable for any IT environment.

### BRIEF DESCRIPTION OF THE SEVERAL VIEWS OF THE DRAWINGS

The foregoing and other features and advantages of the present disclosure will be more readily appreciated as the same become better understood from the following detailed description when taken in conjunction with the accompanying drawings, wherein:

FIG. 1 illustrates a typical hardware environment in the form of a known medical device;

FIG. 2 is a block diagram of the system of the present disclosure in the context of a technician's computer hard-wired to a memory storage device of an ultrasound machine;

FIG. 3 is an external view of the hardware environment of FIG. 2;

FIG. 4 illustrates in more detail the two modules of the tool formed in accordance with the present disclosure;

FIGS. 5A and 5B are a high-level overview of the process employed by the tool of the present disclosure;

FIG. 6 illustrates a method for implementing the process of identifying and resolving error codes by activating a replacement component in the medical device;

FIG. 7 illustrates connectivity components provided in accordance with the present disclosure for use with ultrasound imaging equipment;

FIG. 8 is a flow chart illustrating a method of accessing and retrieving equipment log information in accordance with one method of the present disclosure; and

FIG. 9 illustrates run time operational aspects of a method in accordance with the present disclosure.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures or components or both associated with the repair and replacement of components in medical device, medical imaging equipment, including but not limited to medical imaging equipment such as ultrasound machines, have not been shown or described in order to avoid unnecessarily obscuring descriptions of the implementations.

Unless the context requires otherwise, throughout the specification and claims that follow, the word “comprise” and variations thereof, such as “comprises” and “comprising” are to be construed in an open inclusive sense, that is, as “including, but not limited to.” The foregoing applies equally to the words “including” and “having.”

Reference throughout this description to “one implementation” or “an implementation” means that a particular feature, structure, or characteristic described in connection with the implementation is included in at least one implementation. Thus, the appearance of the phrases “in one implementation” or “in an implementation” in various places throughout the specification are not necessarily all referring to the same implementation. Furthermore, the particular features, structures, or characteristics may be combined in any suitable manner in one or more implementations.

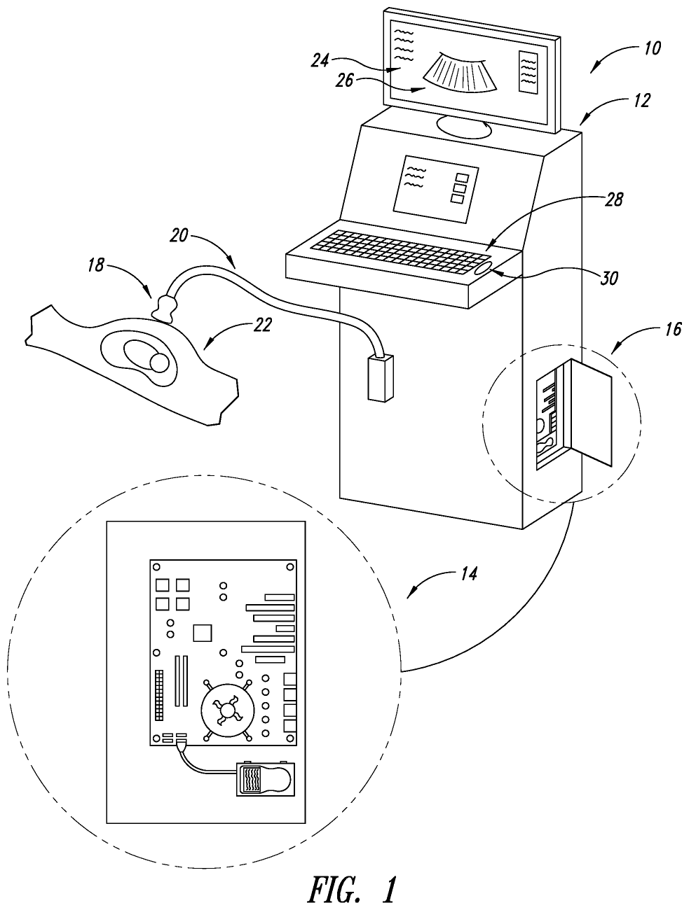

Referring initially to FIG. 1, shown therein is a representative illustration of a hardware environment in which the present disclosure is implemented. A medical device 10 is shown in the form of an ultrasound machine 12. It is to be understood that although the medical device 10 is described in the context of an ultrasound machine, it is intended to embrace all types of computerized equipment, including without limitation medical imaging equipment and related components, such as a transducer head for use with ultrasound machines.

As shown in FIG. 1, the ultrasound machine 12 typically includes a computer 14 or central processing unit (CPU) (shown schematically) that is mounted inside a housing 16 and configured to control operation of the ultrasound machine 12, perform calculations, and control the electrical power supplies for itself and the remote transducer probe 18. The probe transmits pulses or sound waves into the medium of interest, and it also receives the echoes that are converted to electrical pulses, which in turn are sent from the probe 18 to the computer 14, i.e., the CPU. The CPU performs the calculations involved in processing the data and forming an image for display.

The remote transducer probe 18 is coupled to the medical device 12 via a cable 20 and is configured to send the sound waves into a patient 22 and to receive the echoes of the sound waves. A transducer pulse control system is provided (not shown) that enables an operator to change the amplitude, frequency and duration of the sound waves or pulses emitted from the transducer probe 18. A display device 24, such as a monitor, displays the image 26 from the ultrasound data processed by the computer 14. Typically a user interface device, such as a keyboard 28 or mouse 30 or both, is included to input data and commands to the computer 14 and to facilitate taking measurements from the display device 24. Also included is a memory storage device (not shown), such as a disk storage device (hard drive, floppy disk, CD), a solid-state drive, or other types of memory devices that store the acquired images, and an output device (not shown) in the form of a display device or a printer that prints the image or selected aspects of the image from the display device. A remote server may also be utilized to store the acquired images.

Ultrasound has been adopted for a variety of clinical settings, including obstetrics and gynecology, cardiology and cancer detection. A chief advantage of ultrasound is that certain tissues can be observed without using radiation, such as in X-ray equipment. Ultrasound can also be done much faster than X-rays or other radiographic techniques.

However, ultrasound machines are subject to malfunctions and component failures as are other electronic equipment. High temperatures, age, component quality and other factors affect the operational reliability and life span of circuit boards and discrete components on the boards, transducer heads, cables, display devices and associated hardware. Over time, it may be necessary to replace one or more boards and components with new or refurbished components.

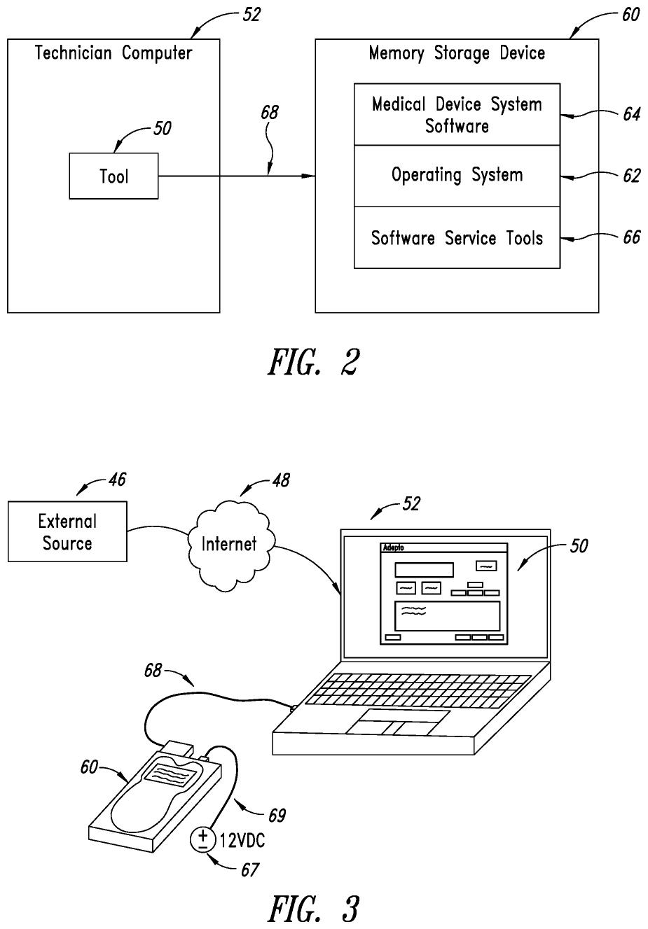

In accordance with the system and method of the present disclosure, a diagnostic and utilities tool, also referred to herein as the tool 50, is provided as shown and described more fully below in connection with FIGS. 2-6.

In accordance with implementation, the tool is a computer-implemented tool for use with a replacement component in a medical device having a computer system. The tool 50, as described more fully below, includes an unsecured software-implemented suite of diagnostic tools and utilities configured to communicate with the computer system of the medical device to read logs, read a bill of materials, provide a Telnet function, eject a USB, and deactivate itself. In addition, the tool 50 includes a secured software-implemented suite of diagnostic tools and utilities configured to interpret a key file received from an external source, upload the tool into the computer system based on the key file, run the tool upon booting up of the computer system, and enable the replacement component in the medical device to be recognized and activated after installation in the medical device.

FIG. 2 is a block diagram of the system of the present disclosure in the context of a technician's computer 52 hard-wired to a memory storage device of a medical device.

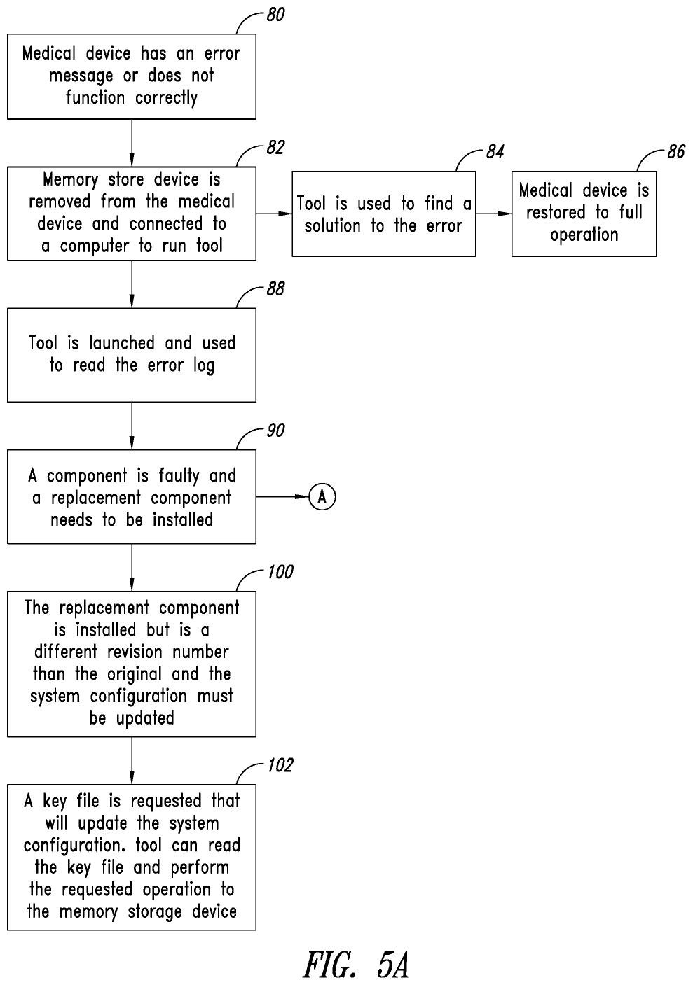

The tool 50 is installed on a technician's computer 52. The tool 50 has two modules, an unsecured module 54 and a secured module 56, both of which are further illustrated in FIG. 4. Briefly, the unsecured module 54 contains an unsecured software-implemented suite of diagnostic tools and utilities configured to read logs, read a bill of materials, provide a Telnet function or other remote communications protocol to communicate with the medical device computer, eject a USB for safely removing the memory storage device from the technician computer 52, and deactivate itself. The secured module 56 is a secured software-implemented suite of diagnostic tools and utilities configured to interpret a key file received from an external source 46, and enable a replacement component in the medical device to be activated, upload at least a portion of the tool into the medical device computer system (e.g., computer 14) based on the key file, run the tool upon booting up of the medical device computer system, and remove or uninstall itself after installation of the replacement component is complete. Uploading a portion of the tool to the medical device may be referred to as uploading a protocol to the medical device based on the key file.

Also shown in FIG. 2 is the memory storage device 60 of the computer 14 on which is installed an operating system 62, medical device system software 64 (e.g., ultrasound imaging software), and software service tools 66 (e.g., firmware), which are coupled to the operating system 62. An external connection 68 is provided, such as in the form of a cable, to enable communications between the technician's computer 52 and the operating system 62 on the memory storage device 60.

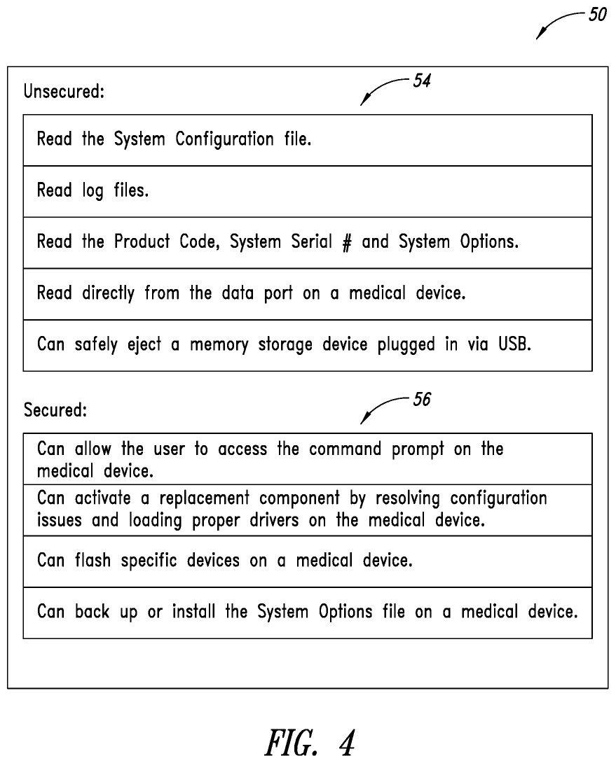

FIG. 3 provides an external hardware view of these connections. Here, the memory storage device 60 is removed from the ultrasound machine 12 and powered by an external source 67 via a power cord 69. For example, the power cord 69 may be plugged into a standard 120V wall outlet and include circuitry to supply the proper voltage to the memory storage device 60. It is to be understood that the hard wired electrical connection between the memory storage device 60 and the technician computer 52 can instead be accomplished via a wireless communication scheme, such as Bluetooth® or Wi-Fi or other known radio frequency communication method. Alternatively, the technician computer 52 can be electrically connected to the memory storage device 60 while it is still in the ultrasound machine 12 via a cable or wireless connection.

The tool 50 receives the key file from external source 46 via the internet 48 or some other network connection. The key file is then utilized by the tool 50 to determine which functions of the secure module 56 to employ. In some scenarios, a license from the external source 46 may be needed before the key file is obtained by the tool 50.

FIG. 4 illustrates in more detail the two modules of the tool 50 formed in accordance with the present disclosure. The unsecured module 54 includes, without limitation, diagnostic software that performs a variety of functions, including reading a system configuration file; reading an error log from the machine's memory storage device; reading the key file, system serial number, and system options; reading information from the front end of an ultrasound machine 12 using a serial port; and safely ejecting a memory storage device plugged in via a USB connection. The secured module 56 has functions that include allowing the user to access the system's command prompt; updating the system configuration and allowing components to be replaced; flashing specific devices on the machine 12; and backing up or installing the systems options for the machine 12.

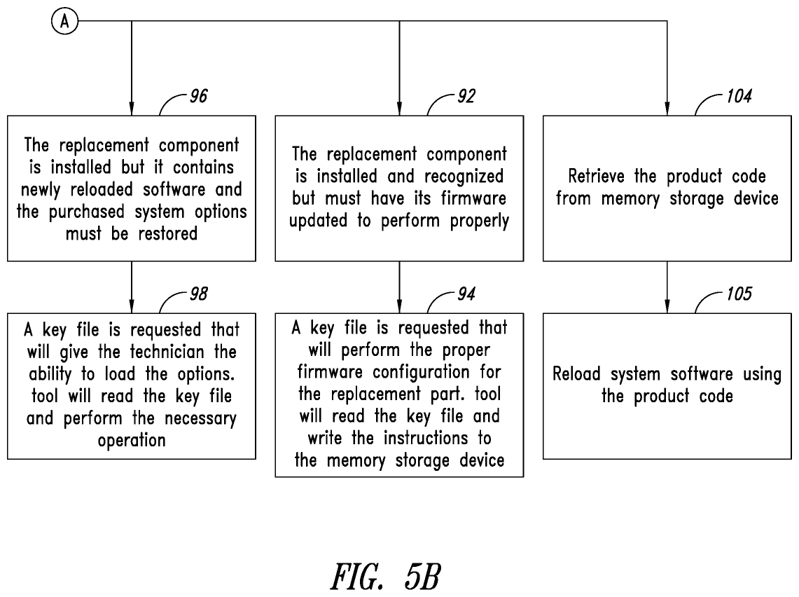

Referring next to FIGS. 5A and 5B, shown therein is a high-level overview of the process employed by the tool of the present disclosure. Initially, at step 80, the medical device has an error message or does not function correctly. The on-site personnel remove the memory storage device from the machine and connect it to the computer in step 82. Once connected, the tool either immediately corrects the error or aids the personnel or a remote technician in correcting the error, as shown in step 84. The machine is then restored to full operation in step 86.

Alternatively, after the memory storage device is coupled to the computer in step 82, the tool is launched and used to read the Error Log (step 88) of the machine. If it is determined that a component is faulty and a replacement component needs to be installed in step 90, one of four things can happen.

First, the replacement component is installed and recognized but must have its firmware updated to perform properly (step 92), after which a key file is requested that will allow the tool to perform the proper firmware configuration for the requested replacement component. The tool will write the corrective instructions to the memory storage device in step 94 to update the firmware and enable the replacement component to properly function.

Second, the replacement component is installed but it contains newly reloaded software and the purchased system options must be restored (step 96). This is done in step 98 by requesting a key file, which will give the technician the ability to load the options on the replacement component. The tool will read the key file and perform the necessary operation load operation.

In a third scenario, the replacement component is installed but is a different revision number than the original component and the system configuration must be updated (step 100). Again, a key file is requested that will update the system configuration in step 102. The tool reads the key file and performs the requested operation to the memory storage device to enable the replacement component to perform its function within the system.

In a fourth scenario, the replacement component is installed but the system software needs to be reloaded. The product code is retrieved from the memory storage device at step 104, and the system software is reloaded on to the replacement component using the retrieved product code at step 105. The replacement component is then able to function in accordance with design specifications within the context of the medical device in which it is installed.

After the tool performs the appropriate functions on the memory storage device, the memory storage device is reconnected to the medical device. The computer on the medical device is rebooted, and the tool functionality uploaded on the memory storage device can execute, which includes removing the tool from the memory device.

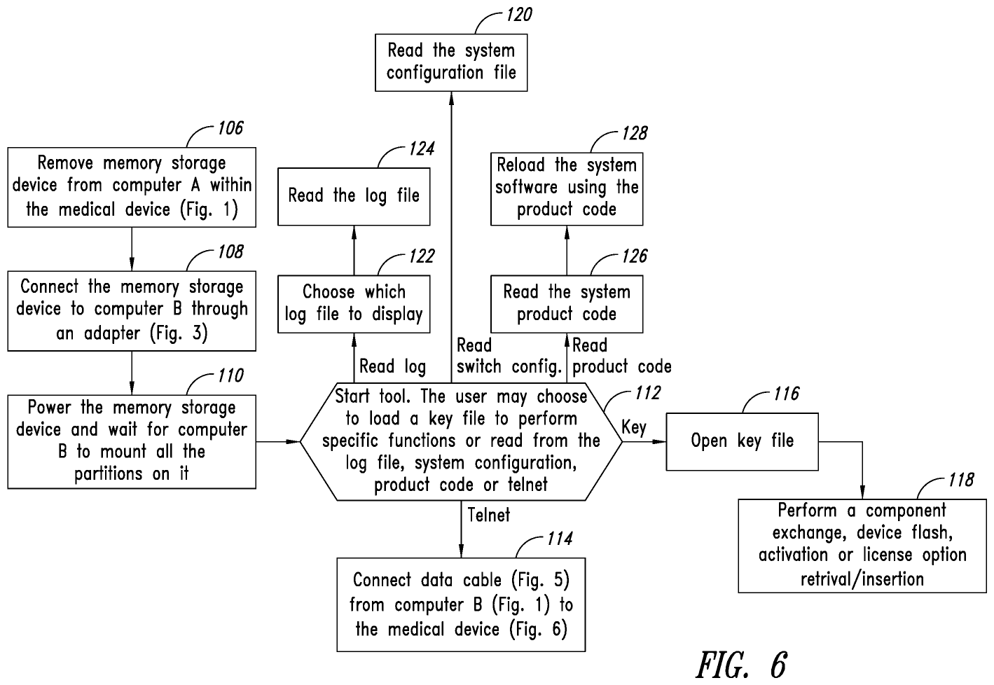

FIG. 6 illustrates a process diagram of the method for implementing the process of identifying and resolving error codes and activating a replacement component in the medical device. In an initial step 106, the memory storage device is removed from the medical device and is connected to a USB adapter and power is supplied by a power adaptor. The USB connector is connected to the technician's computer in step 108, and power is applied to the memory storage device. The tool on the technician's computer then accesses and recognizes the memory storage device.

The technician then starts the tool in step 112. It is at this point that the technician may choose to load a key file to perform specific functions or read from the Log or System Configuration or Telnet. The functionality available to the technician is, for example, shown in FIG. 4. For example in step 114, if Telnet is used, the cable is coupled to the serial port or other suitable port on the medical device and the unsecured module only is available to the technician.

Alternatively, in step 116, the key file is opened, and then the tool is used to perform a device flash or product activation as shown in step 118. This is the secured module of the tool as described above with the functionality shown in the table in FIG. 4. These steps can only be done if the memory storage device is connected to the technician's computer.

If the memory storage device is removed from the medical device, then the process shown in steps 120, 122, and 124 would be implemented. More particularly, using the unsecured functionality of the tool, the system configuration file can be read in step 120. Or, the Error Log can be read wherein the technician can choose which Error Log to display in step 122 and to read it in step 124. Or, the product code can be read in step 126 from the memory storage device, and the system software is reloaded at step 128 using the product code.

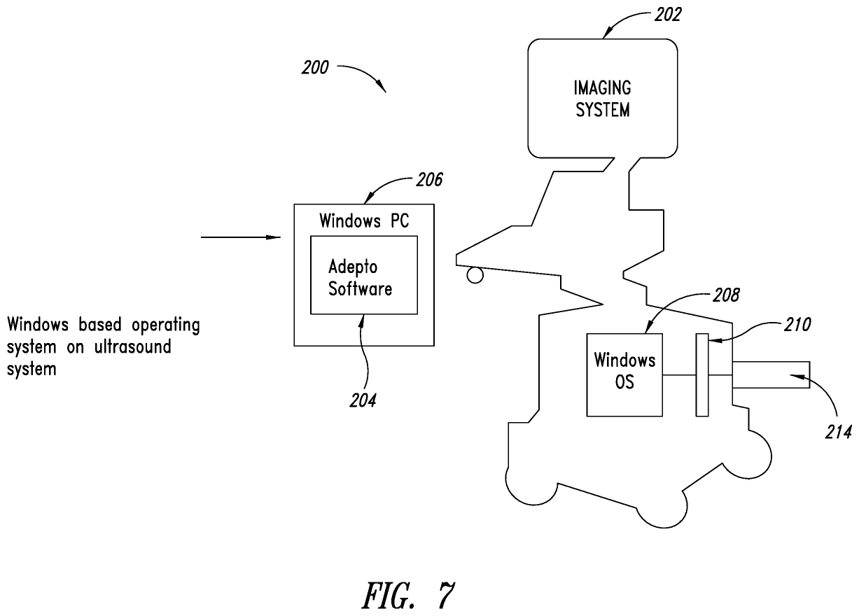

Referring next to FIG. 7, shown therein is a system 200 to enable remote connectivity and access to logs on electronic imaging equipment, in this case an ultrasound imaging machine 202. The system as broadly shown therein includes a software tool 204, identified as “Adepto®,” installed on a Windows® PC 206, which is configured to communicate with the ultrasound imaging machine 202 either wirelessly or via a direct wired connection. The ultrasound imaging equipment 202 includes a Windows-based operating system 208 that accepts a variety of hard-wired connections, including a USB port 210 that accepts a USB flash drive 212. Stored on the flash drive 212 is an executable software application 214 that is configured to access equipment logs on the ultrasound imaging machine 202, parse the equipment logs for equipment operating information and condition information, and retrieve this information as electronic data. Ideally the data is transferred to a user interface, either on-site or off-site, either wirelessly or via a wired connection.

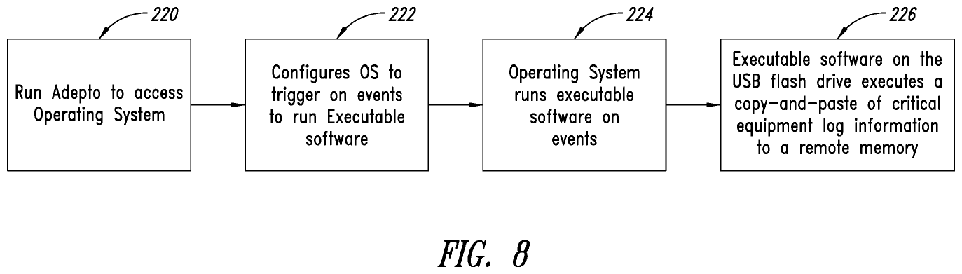

FIG. 8 illustrates a high level process for using the executable software. As shown, the first step 220 is initiation and running of the Adepto software 204 that, among other things, in a second step 222 communicates with the Windows OS 208 on the ultrasound imaging machine 202 and configures the Windows OS 208 to trigger the executable software application 214 on the flash drive 212 in response to the occurrence of certain events. Triggering events can include elapsed time, such as every 10 seconds, every 15 seconds, every 20 seconds, every 30 second, every minute, every 6 minutes, every 60 minutes, and other time intervals based on determinations by the operator as well as the nature and usage of the equipment.

In a third step 224, in response to occurrence of a triggering event, the executable software application 214 accesses the equipment logs on the ultrasound imaging machine 202. In a fourth step 226 the executable software application 214 copies equipment logs pertaining to equipment operating information and equipment condition information, which data is then transmitted to an on-site or off-side storage device. The storage device can then be associated with a user interface that displays the data, which can be used to service the ultrasound imaging machine 202 using the protocols and tools described herein.

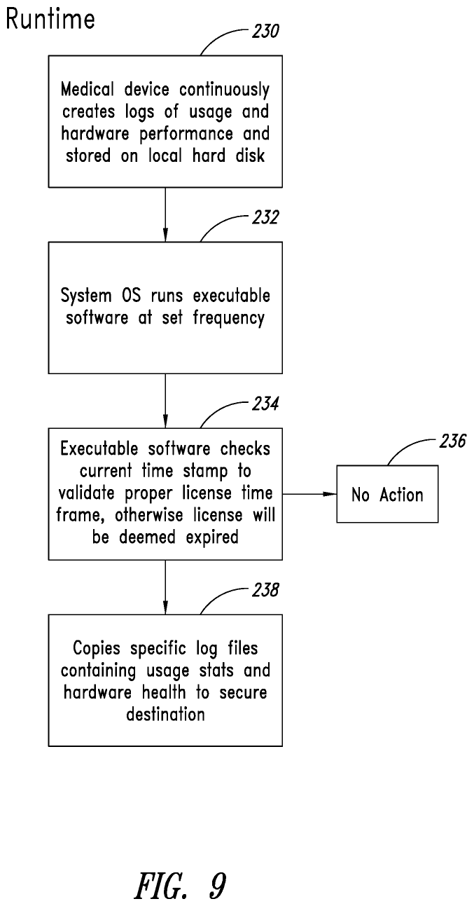

FIG. 9 illustrates run time operational aspects of a method in accordance with the present disclosure. In box 230 is shown the creation of logs of usage and hardware performance that is stored on a local hard disk by the medical device, such as the ultrasound imaging machine 202. This creation and storage is done by the operating system and on-board software of the medical device.

In box 232 the operating system (OS) of the medical device, such as the ultrasound imaging machine 202, runs executable software at a set frequency as discussed above. In box 234 the executable software on the USB drive accesses the medical device. An optional license verification step is performed that initially checks to determine if the owner or operator of the medical device has a current license to use the executable software. If not, the process stops in box 236.

In response to confirmation of a current license, the executable software makes copies of specific log files that contain usage statistics and hardware health and saves this information or data to a secure memory location outside of the medical device. The data can then be utilized in a user interface, such as a dashboard or similar display for analysis and action as needed to maintain the medical device in an operational status, make necessary or recommended adjustments and repairs, and generate reports on the equipment and specific components that enables analysis regarding trends, reliability, and anticipated repairs.

The various implementations described above can be combined to provide further implementations. Aspects of the implementations can be modified, if necessary to employ concepts of the various patents, applications and publications to provide yet further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A computer-implemented tool for use with a replacement component in a medical device having a computer system, comprising:
an unsecured software-implemented suite of diagnostic tools and utilities configured to communicate with the computer system of the medical device to read logs, read a bill of materials, provide a Telnet function, eject a USB, and deactivate itself; and
a secured software-implemented suite of diagnostic tools and utilities configured to interpret a key file received from an external source, upload the tool into the computer system based on the key file, run the tool upon booting up of the computer system, and enable the replacement component in the medical device to be recognized and activated after installation in the medical device.

2. The tool of claim 1, wherein the secured software-implemented suite of diagnostic tools and utilities is configured to remove or uninstall itself from the computer system and restore a file structure back to original equipment manufacturer standards.

3. The tool of claim 1, wherein the tool includes a communication circuit configured to communicate with the external source to receive the key file.

4. A system, comprising:
a medical device that includes:
a controller having a control circuit and a memory in communication with the control circuit; and
a plurality of components in communication with the controller, the plurality of components including at least one replacement component; and
a remote device capable of being in communication with the memory of the medical device and configured to communicate with the memory to enable the medical device to activate the at least one replacement component, the remote device including:
a memory having stored thereon a plurality of unsecured diagnostic and utility programs and a plurality of secured diagnostic and utility programs that are configured to be activated upon receipt of one or more key files;
a software program configured to receive the one or more key files to enable the use of secured functions of the secured diagnostic and utility programs to provide an enabling code to the memory of the medical device to be used to activate the at least one replacement component.

5. The system of claim 4, further comprising a remote provider configured to communicate with the remote device and provide the one or more key files to the remote device.

6. A method of a recognizing and activating a component in a medical device, the method comprising:
receiving a key file from an external source;
uploading a protocol to a memory of the medical device;
running the protocol as enabled by the key file to enable the activation of one or more replacement components; and
removing the protocol from the memory of the medical device after the one or more replacement components is activated.

7. The method of claim 6, further comprising:
reading an error log to identify and interpret error codes in the error log and identify the one or more replacement components in the medical device that are not activated; and
clearing the error log based on the key file.

8. A method, comprising:
accessing an operating system on an electronic equipment;
configuring the operating system to run an executable program when triggered by one or more events;
executing the executable program to access equipment logs; and
copying equipment log information to a second location for display on a user interface.
